(19) United States
(12) Patent Application Publication
(10) Pub. No.: US 2019/0030336 A1
(43) Pub. Date: Jan. 31, 2019

(54) Portable Composite Waveform Transcranial Electrical Stimulation System

(71) Applicant: Pui Tong Kwan, Hong Kong (CN)

(72) Inventor: Pui Tong Kwan, Hong Kong (CN)

(21) Appl. No.: 16/147,515

(22) Filed: Sep. 28, 2018

(51) Int. Cl. A61N 1/36; A61N 1/04; A61N 1/02

(52) U.S. Cl. CPC A61N 1/36025; A61N 1/0456; A61N 1/025; A61N 1/36034; A61N 1/0476

(57) ABSTRACT

The present invention relates to a transcranial electrical stimulation (TES) system in that several sets of manipulated, pre-stored raw data are executed. TES signals are generated and output to user in terms of sessions. The output waveform in each singular session is a composite type and is designed mainly by means of Fourier Transform basis together with numerical analysis and mathematical curves fitting techniques. Hence, the electrical signals' frequencies, amplitudes and phase angles are varying during the time of application within just ONE singular session by one device. The kinds of variations in parameters such as frequencies, amplitudes and phase angles are tailor-made to the needs of improvement required in several brain disorders caused or related neurological disorders symptoms. With the help of the wearing design, the brain can be stimulated in dedicated positions such that the TES signals are continuously monitored by the unit.

### BACKGROUND OF THE PRESENT INVENTION

### Field of Invention

The present invention relates to a technical field of electrotherapy, and more particularly to a portable composite waveform Transcranial Electrical Stimulation (hereinafter called TES) system. It is by no means impossible to be further elaborated from an inexpensive portable device into a sophisticated type of equipment in professional application.

The whole TES system comprises a microprocessor as the main functional unit, as shown in FIG. 12. The firmware is resided into the flash memory internally in microprocessor. Other peripheral electronics modules are applied to different functions. “Impedance Tracking Module” caters for continuous monitoring of outputs' characteristics as well as device's connectivity. Controlled output—“Voltage-Current Correction” is made by means of feedback loops based on impedance information. “Time Control Module” caters for event logs and precise timer control. “USB Interface Module” caters for file system handling, communication protocols for “DATA Input and Output” as well as charger power input.

The power management of portable TES system consists of “Battery Backup Unit”, “Battery Charge Control Unit”, “Battery Protection Unit” and a “Fuel-Gauge Unit” that provides a remaining battery capacity reference information for user. A “Handheld Remote-control Unit” is provided for easy operation of the device when one is wearing the device on his head. Either 2.4 GHz, Bluetooth, or Infra-red type wireless communication protocols is used in the remote-control unit.

A simplified version of the portable composite waveform TES system “Without Remote-control” is also available. The major difference is the release of TES signal will be triggered by the On/Off switch of the headset instead of the remote-control.

A “Commutative Probe System” consists of multi-electrodes (channels No. 1 to 8) serves to provide commutative electrical stimulation signals that applied to different relative spatial positions of brain's regions. The device itself has features of “Auto Sleep Mode”, “Touch Switch ON-OFF Module”, “Auto-Detection for Disconnections” between Probe system and the user's head.

Several sets of experimentally verified data are pre-stored into the external flash memory of device by downloading via the personal computer. The main stimulation programs and hence the profiles of composite electrical waveforms are generated in microprocessor, exported to the “Digital to Analog Processing Unit” for conversion, amplified by the “Signal Amplifier Unit” and final TES signals was sent to the “Commutative Probe System”. The composite waveform TES signals that contains variations of frequencies, amplitudes and phase angles are output to user. These variations of parameters can be tailor-made according to the needs of improvement required in several brain disorders caused or related neurological disorders symptoms.

Moreover, these parameters can also be custom-made according to individuals' needs when it is required upon request for firmware re-program or upgrade through internet connection. The output composite waveform type electrical signals can be “Alternating Current” (AC) voltage type or “Direct Current” (DC) voltage type and is proven to have prominent improvement in the symptoms mentioned as below.

Over a hundred articles and studies regarding the clinical application of Transcranial Electrical Stimulation (TES) technology (Transcranial Direct Current Stimulation, tDCS and Transcranial Alternating Current Stimulation, tACS) on neurological disorders are available for reference:
Insomnia (Rivera-Urbina et al. 2016 J. Sleep Med. Disord. 3-1060),
ADHD (Breitling et al. Front. Cell. Neurosci. 2016, 10:72),
Autism (Amatachava et al. Behavioural Neurology Vol. 2014, Article ID 173073),
Alzheimer's Disease (Bystad et al. Alzheimer's Research & Therapy 2016, 8:13).
Parkinson's Disease (Hendy et al. Trials 2016, 17:326),
Depression (Loo et al. BJPsych 2012, 200, 52-59),
Stroke (Lefebvre et al. Front. Neurol. 2017.00029), etc.

### Description of Related Arts

The statements in this part only provide background information relating to the disclosure of the present invention and may not constitute a prior art.

Electrotherapy is not a new technical idea. Aristotle and Plato have mentioned the Torpedo fish recorded by a physicist Scribonius Largus in AD (Anno Domini) 46 years, which was able to alleviate a variety of diseases from headache to the pain, from the headache to gout. In the 18^{th} century, dentists also proposed a method which adopted an electronic treatment instrument to alleviate pain. Until the end of the eighteenth century, electronic devices have been widely used to control pain and to conduct treatments on a variety of diseases in medical fields.

In 1902, Leduc and Rouxeau doctors in France firstly reported an experiment which the micro-current was used to stimulate the brain. The main result of the experiment was to generate hypnotic effect, and later the experiment was further applied to control anxiety, insomnia, depression and pain. The treatment method has also an early name called Cranial Electrotherapy Stimulation (CES). In 1965, Ronald Melzack Doctor in Canada and Patrick Wall Doctor in the United Kingdom published a paper in which a comprehensive theory was written to explain pain formation in the nervous system. Their Gate Control Theory also explained how electronic stimulation affected the pathways of pain based on physiological principal. The theory was further applied to Transcutaneous Electrical Nerve Stimulators (TENS) and other related devices.

All lives possess the essence of electrochemistry, and similarly, the body's nervous system can also work through electrochemical signals, and simply through electrical signals. Therefore, neurologists are continuously studying on how to cure and prevent neurological diseases through human body electrophysiology regulations. Recently, neurological diseases have become a major threat to human health and quality of life today. In accordance to the global aging trend, the nervous system diseases and the sequelae of the diseases have been accounted for a large portion of the national medical and health expenditures in worldwide. Whereas the neurological diseases related to brain are increasingly rising in ratio, the main medical therapy methods include medication, surgery and psychological counseling which have their own drawbacks. Amount from these, an invasive therapy method, i.e., Deep Brain Stimulation (DBS) is developed, in which electrodes are implanted in a specific region of the brain by surgical means to mitigate the symptoms of the diseases through specific electrical stimulations. However, the method is an invasive treatment method which possesses high technical difficulty and risky characteristics. In the direction of non-invasive technical methods, two means including “Transcranial Magnetic Stimulation” (TMS) and “Transcranial Electrical Stimulation” (TES) are developed. However, the TMS method's device requirement as well as operator's professional requirement is higher. The TES method is safer in operation, smaller side effects and easier to operate so that it has become the heat topic of neurologists in last two decades.

In existing market, the main operation modes (electric current) of TES devices include Pure Direct Current (DC), single frequency Alternating Current (AC), DC pulses, composite frequencies of pulses or AC, Random Noise (RN). Mostly, they are the single-mode electrical stimulation devices. There are also devices that integrate modes of operations as mentioned above. Some of the devices possess function on several parameters adjustment and some have the Electroencephalograph (EEG) signals acquisition function.

Currently, the TES devices that exist in the market have the following inadequacies in performance:

(1) The electric current output mode is unique during the stimulation process. The common modes of operations in TES devices on the market are namely, pure Direct Current (DC), Alternating Current (AC) (can be chosen for pure sine waves, pulses, triangular waves and asymmetric waveforms), noise mode (low-frequency, medium-frequency, high-frequency, white noise and randomly generated forms). However, only one of the modes can be selected for current output. Furthermore, corresponding parameters of individual waveform such as time-span, peak value of current and frequency can only be preset before starting electrical stimulation. It is deprived of frequency variations and unable to provide a high frequency range. Once the stimulation process begins, the output current waveform will be delivered to the patient continuously without variations in according to the preset configuration of parameters.

(2) Against different kinds of applications such as depression treatment, insomnia treatment and improving cognitive function, there are several kinds of TES devices on the market. These devices usually have a unique form of product type appeared on the market with a built-in fixed program for electrical stimulation and adopt simple parameters adjustment function such as time and intensity as their product solutions and are unable to achieve the import function for a personalized stimulation program.

(3) The TES and the EEG acquisition devices are differentiated into different products and distributed in standalone function on the current market. There are individual devices which integrate the TES and the EEG acquisition functions. However, the TES and the EEG acquisition functions themselves are still separated. EEG data signals are unable to enroll into stimulation process. Thus, the output signals of the EEG stimulation are unable to be adjusted dynamically in accordance with different EEG level.

The present technology needs to be improved and further explored.

### SUMMARY OF THE PRESENT INVENTION

The brain is a complicated organ of human body. It is protected by the scalp at its outermost surface and is then covered by a hard skull bone. The whole brain is suspended in cerebrospinal fluid, and isolated from the bloodstream by the blood-brain barrier. The EEG signals indeed is a resultant of numerous neurons' spiking activities, originated deep inside the brain, that are synchronously aligned. The EEG signals are identified as different ranges oscillation frequencies and are associated with different states of brain's functional activities over a network of neurons in specific spatial distributions inside the brain. The EEG signals themselves are measurements taken across the potential differences variations between two points on the scalp over a time span. Due to the inevitable built-in impedances imposed onto and throughout the pathways (from scalp to deep inside brain), signals received are severely attenuated when compared with the generated sources deep inside the brain from which they are originated. Reciprocally, it is also difficult to overcome the inherent impedance when one wants to conduct TES between two points of the scalp by placing two electrodes on the scalp and applying on voltages. Anyway, whenever there is a conduction after a potential difference applied on the scalp, there exist a resultant impedance.

This resultant impedance, for easy understanding, can be expressed as:

Z_{TOTAL}=Σ(Z₁+Z₂+Z₃+ . . . Zₙ),

wherein:

n denotes the pathway number,

Z₁, Z₂, Z₃, . . . Zₙ are denoted as the individual pathway's impedance, and

Z_{TOTAL} is the summation of vectors in total.

These impedance values are frequency dependent. The basic constituents of impedance are capacitive, inductive and resistive types. All these impedances' arithmetic operations are using vectors. For resistive type, it obeys Ohm's Law and nothing related to frequency. For inductive type, impedance increased with frequency. For capacitive type, it decreases as frequency increases. Therefore, when dealing with TES operations, frequency consideration is inevitable. Without providing an easy platform for handling frequency variations, it is difficult to tackle with the impedance issue arises during TES.

As a matter of fact, the electrical conductivity and hence the impedance from scalp to deep inside the brain is non-uniform. Several pathways for micro-currents will be created depending on the instantaneous state of brain (and hence the resultant impedance) once the electrodes are attached to the scalp of recipients. If, for instance, the applied TES is also having the characteristics of variations, then the pathways are changing according to the state of brain as well as to the varying parameters of the stimulating signals.

The present invention has solved several technical issues by providing a solution as a portable composite waveform transcranial electrical stimulation system. They are as follows:

(1) A new TES system device in which its working principle is by means of transmitting electrical stimulation signals in the form of a composite waveform such that the recipient can experience electrical stimulation signals in terms of amplitudes, frequencies (both in the high range and low range) and phase angles variations.

(2) The composite waveform can be an offset type AC signals or differential AC signals or DC type signals.

(3) A breakthrough is made on bringing up the oscillation frequencies of signals applied and can be adjusted to the extent of 40 kHz in the high side and down to 0.001 Hz in the low side. A better approach can easily be implemented by employing a stimulation profile within ONE stimulation session that possess all required variations of amplitudes, frequencies & phase angles. The kind of waveform (as shown in FIG. 13A) can be a guided envelop of low frequencies (as shown in FIGS. 13B and 13C) with numerous high frequencies super-imposed onto the envelope.

(4) Combining the ability to vary in the amplitudes and the phase angles, it is feasible to simulate a dedicated stimulation profile under customized requirements such that the stimulation profile suits the needs of individual recipient's preference.

(5) By comparing abnormal patients' waveforms with the normal counterparts, which were obtained collectively in practical fields, a generalized curve simulation of the corresponding corrective composite waveform is derived by means of numerical analysis and curves fitting mathematical techniques.

(6) The composite waveform is further smoothened by removing some possible abrupt changes or spikes in voltage such that the pain feelings of puncture or pricking can be avoided when final electrical stimulation signals is applied onto the recipient's scalp.

(7) Manipulated data in the form of file is downloaded into the external flash memory of the TES system device via USB bus. A large storage size of external flash memory is employed. Users can replace their own preference profiles altogether or partially depending on the built-in memory size of the TES system device.

(8) The whole TES system device is powered up by rechargeable battery backup. A built-in charge control unit, battery protection & fuel-gauge are included into the power management. The whole battery system is recharged via the USB power supplies. The power consumption is extremely reduced such that it could last for at least 2 hours after fully charged when compared with conventional TES products. It is not required for AC mains supply. Thus, the present invention of TES system device is no longer a bulky equipment.

(9) Once the device is initialized, the micro-processor inside will execute the control programs for electrical stimulation signals generation. The data (which pre-stored inside external memory locations) will be fetched, interpreted and transformed into a stipulated electrical signals profile—dedicated composite waveform using high resolutions of digital to analog.

(10) The electrical signals are further amplified, regulated and continuously monitored to a safety level by voltage-current correction before they are transmitted as outputs.

(11) The device contains an impedance measurement unit. Real-time measurements will be taken due to possible changes of impedance will happen along the pathway of conducting micro-currents. The measured values are acquired and feedback to the micro-processor for adjustment such that the output amplitude of DC equivalent is ensured to be under control. By means of voltage control corrections, the outcome TES electrical signals' voltage and current are always controlled below a safety magnitude of voltage at 35V and current at 10 mA.

(12) Based on impedance measurement, the device can distinguish whether the user is wearing the device on head properly. In case if there is any loosening in contacts when the TES system device is delivering output, the micro-processor will detect and send out alarm signals to alert user.

### BRIEF DESCRIPTION OF THE DRAWINGS

FIG. 1 shows the front view of the whole set of TES system. In which, the electrical stimulation signals are output to the recipient via a delicate electrical probes system having 4+4 electrodes resided on the left and right side of the headset. The eight positions are relative to the brain's functional 3D spatial regions, namely, Cerebellum, Frontal and Temporal Lobe.

FIG. 2 shows the front view of the whole set of TES system.

FIG. 3 shows the back view of the whole set of TES system.

FIG. 4 shows the front view and the relative positions of electrodes to the brain's functional regions when the headset is put on. Note on the center of gravity.

FIG. 5 shows the back view and the relative positions of electrodes to the brain's functional regions when the headset is put on. Note on the center of gravity.

FIG. 6 shows the brain's functional regions.

FIG. 7 shows two pairs of claws in the upper end that cater for the Frontal Lobe stimulations. Each pair can be rotated in an angle of +/−25 degree along the axis independently to each other and to the low end. There is one pair of claws located in the lower end that cater for Cerebellum of the brain's region. The two claws can be rotated independently in a manner which similar to the upper end but with +/−12 degrees of rotation only. The electrodes that cater for Temporal Lobe are not movable. With enough freedoms of angles of rotations, one can adjust the probes system into their own favorable positions easily.

FIG. 8 shows the positions of sponge, silicone rubber (non-metallic conductive material) and On/Off with status LED.

FIG. 9 shows how the claw-shaped probe system is turnable and removable.

FIG. 10 shows the location of the main functional unit in the headset and how it is embodied inside a detachable compartment as depicted.

FIG. 11 shows the individual components and the assembly of the claw shape probe.

FIG. 12 shows that the whole TES system comprises a microprocessor as the main functional unit.

FIG. 13A shows a general composite waveform.

FIG. 13B shows that the waveform is enlarged in 100 times view in Region 1.

FIG. 13C shows it is further enlarged in Region 2 of the curve in Region 1.

### DETAILED DESCRIPTION OF THE PREFERRED EMBODIMENT

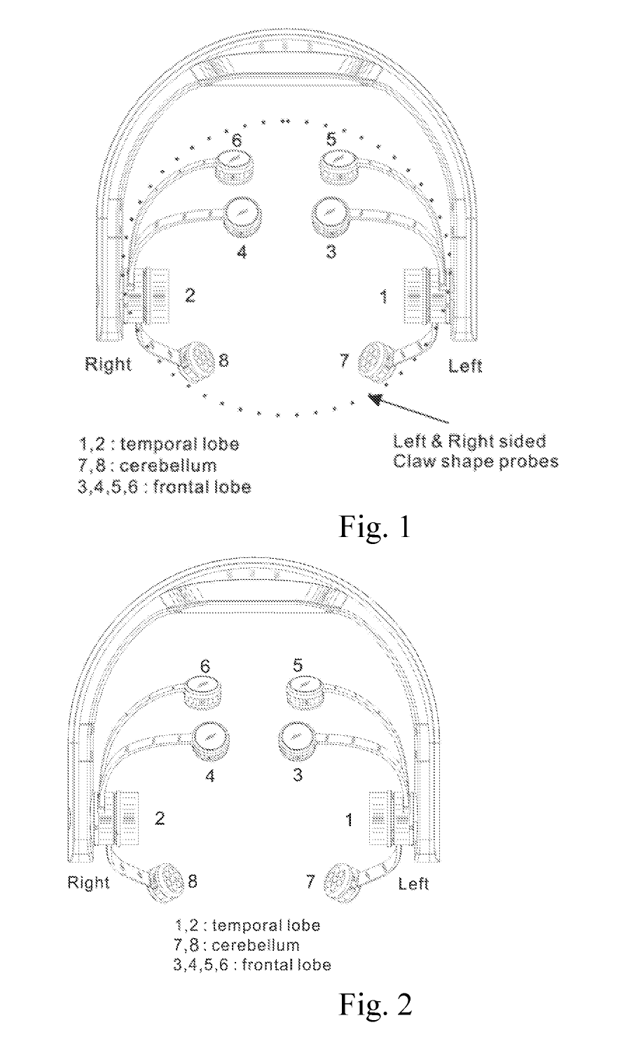

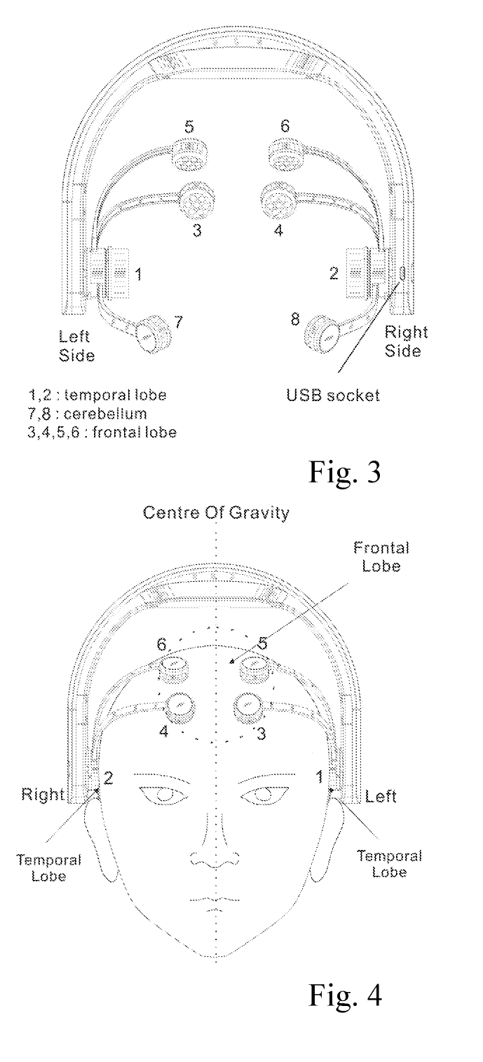

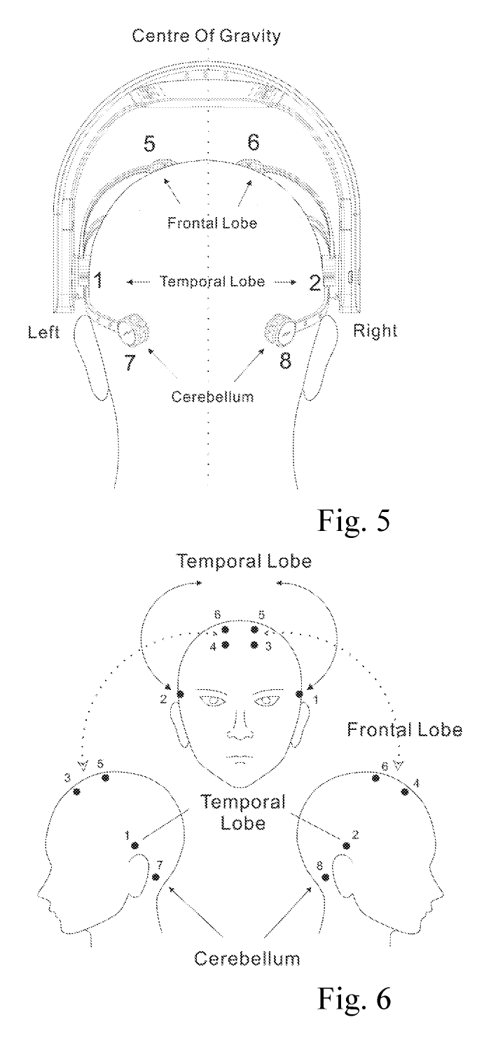

The electrical stimulation signals are output to the recipient via a delicate electrical probes system (FIG. 1) in which electrodes are resided into relevant positions. A total of eight individual electrodes are equipped in the headset as shown (FIG. 2: Front View, FIG. 3: Back View). When the headset is put onto the head as shown in FIG. 4 and FIG. 5, the probes system's structural design is made such that the orientations of the electrodes' contacts are located at the relative spatial distribution of brain's functional regions as shown in FIG. 6.

Once TES signals arrived onto the recipient's scalp surface, numerous pathways carrying different micro-currents are created. The created pathways and their current magnitudes are depending on the snap shot of that instantaneous state of brain as well as influenced by the composite waveform's varying parameters, i.e. amplitude, frequency and phase. These pathways do not exist persistently. They are all developed according to states of conditions, i.e. states of brain and composite waveform varying parameters. Whenever changes in the states of conditions are, new sets of conductive pathways will be created. Due to these variations over the time span, a complex network of conducting pathways will be created in a 3D manner over the probed regions across the applied electrodes. The electrical stimulation signals are easily scattered and spreading out under the composite waveform's electrical potential throughout the intended regions of the brain.

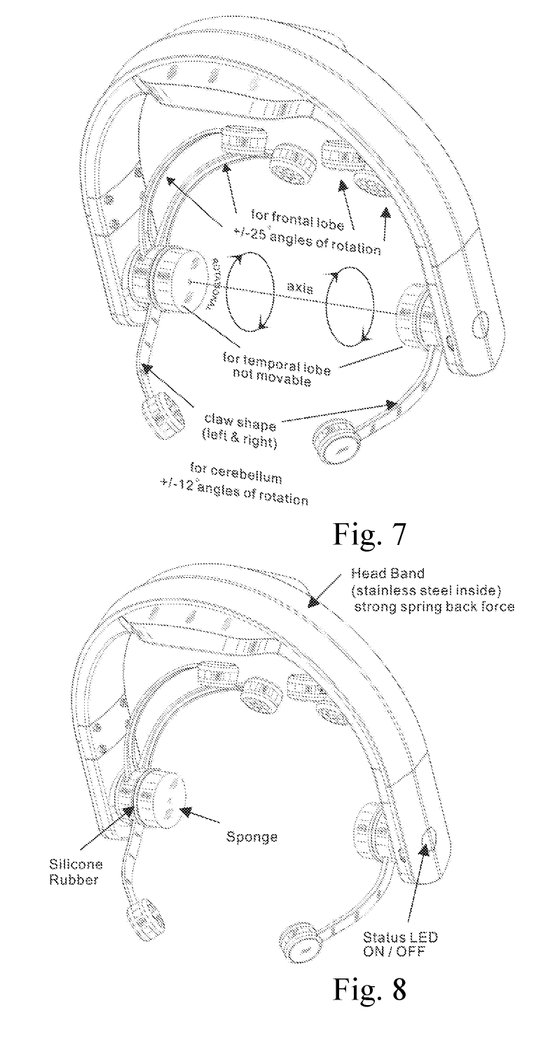

The probes system's structural design follows tightly in the knowledge of Ergonomics. It allows us to access and contact onto the targeted functional region of the brain (see FIG. 6). The center of gravity (COG) of whole structure (see FIGS. 4 & 5) is aligned on the symmetric line of human beings and users can rotate and adjust the probes system in their favorable positions (FIG. 7). There is a strong stainless steel embedded inside the headband that provides an inwards spring back bending force when users put on the device. It is important to ensure a secure mounting, support and right positioning of probes system. The conductivity between the contact surfaces of the probes and scalp surfaces can be further enhanced with the help of conductive silicone rubber and sponge soaked in brine water (FIG. 8). To achieve the best conductivity, sponges can be installed on all 8 electrodes for patients with long hair. Or, only the conductive silicone rubber can be used for the bare head.

Multi-channels of electrical stimulation and flexible selection of electrode pairs are available. Depending on the needs, users can program the device to stimulate more than one region of brain intermittently by means of commutated pairs of electrodes within chosen probes via programming. Different combination of stimulating output electrodes (channels/electrode pairs) can be programmed as follows:
(a) Electrode No. 1 and Electrode No. 2,
(b) Electrode No. 3 and Electrode No. 4,
(c) Electrode No. 5 and Electrode No. 6.
(d) Electrode No. 7 and Electrode No. 8,
(e) Electrode No. 1 and Electrode No. 4,
(f) Electrode No. 1 and Electrode No. 6,
(g) Electrode No. 2 and Electrode No. 3.
(h) Electrode No. 2 and Electrode No. 5,
(i) Electrode No. 1 and Electrode No. 2,
(j) Electrode No. 1 and Electrode No. 7,
(k) Electrode No. 2 and Electrode No. 8, etc.

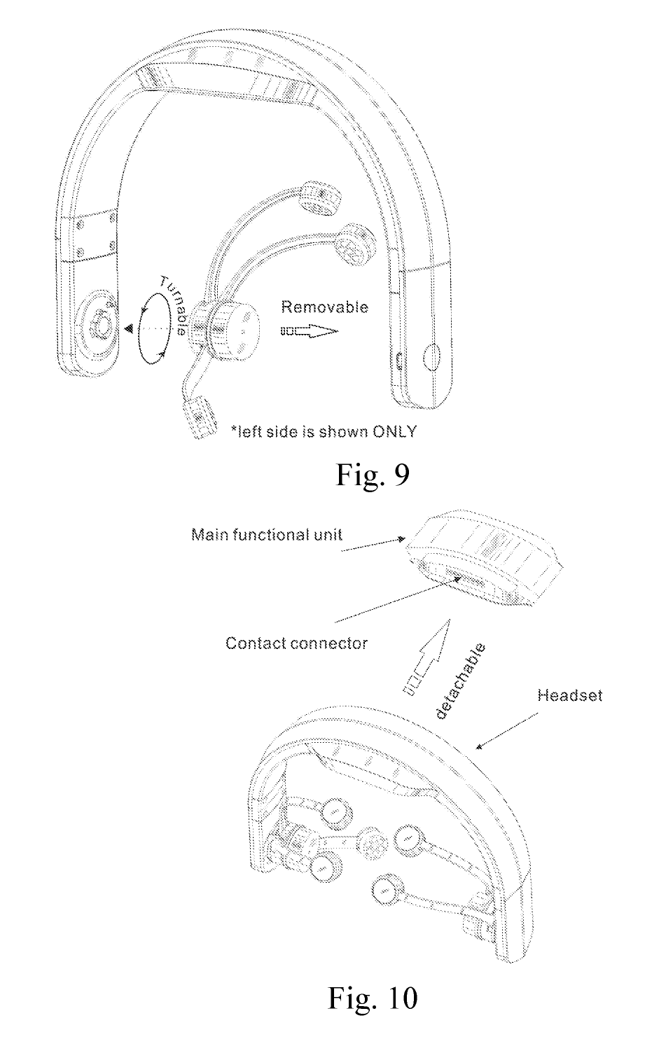

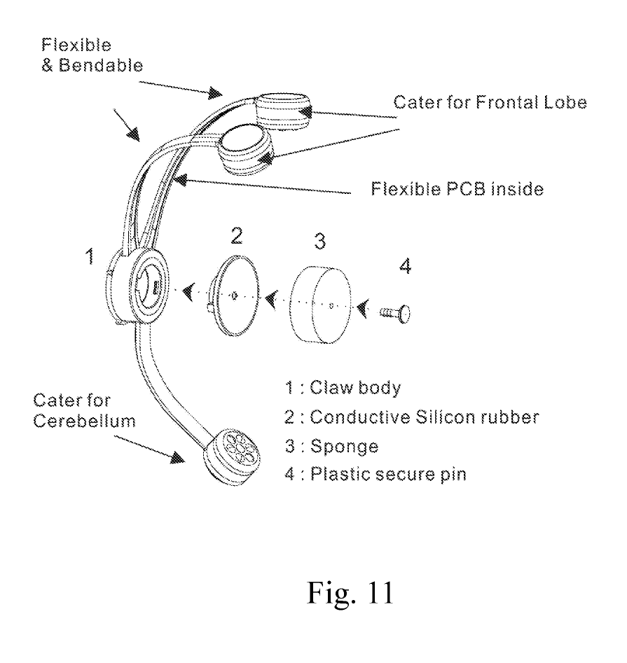

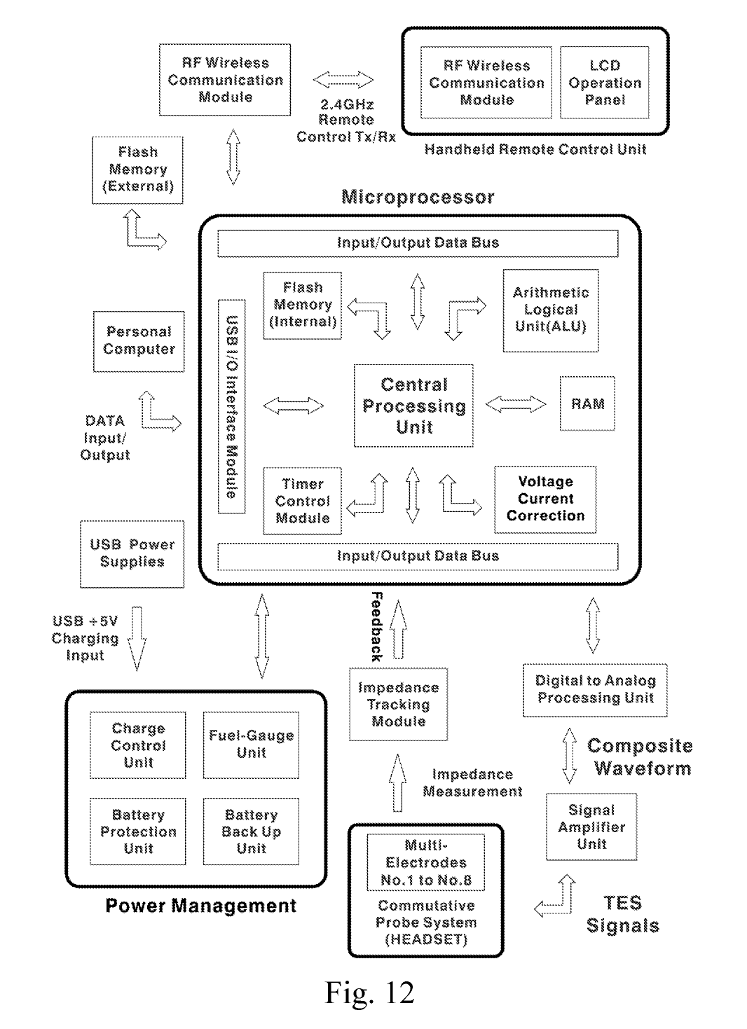

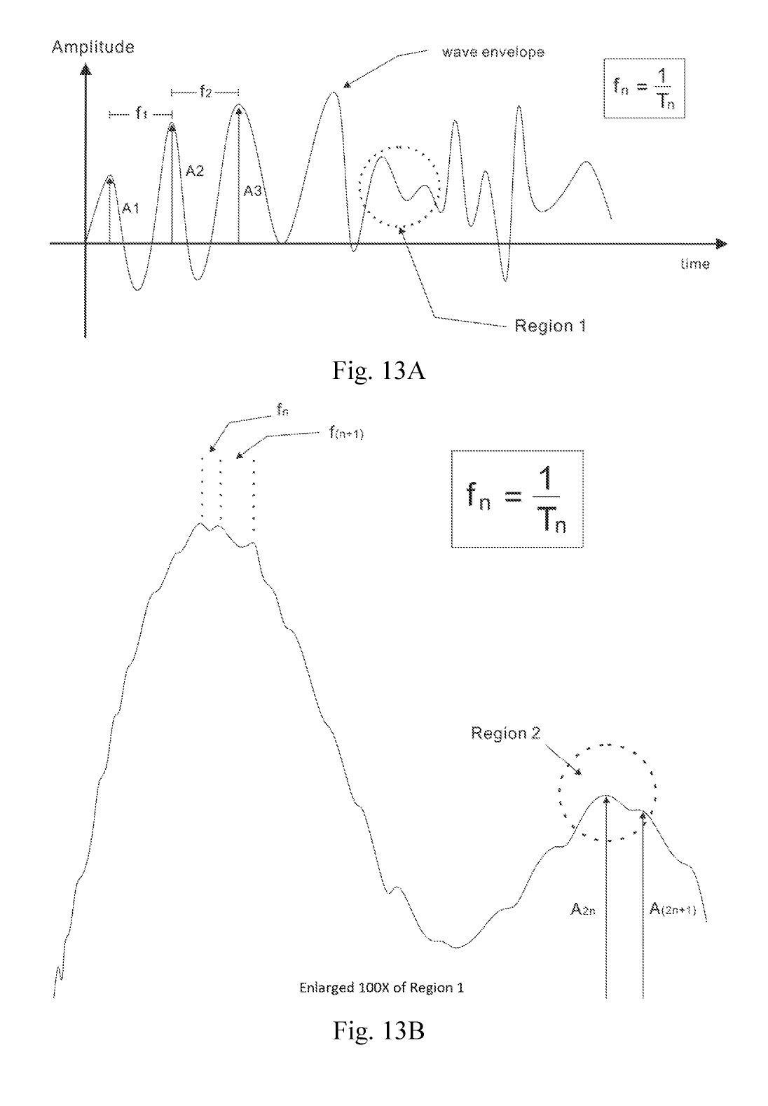

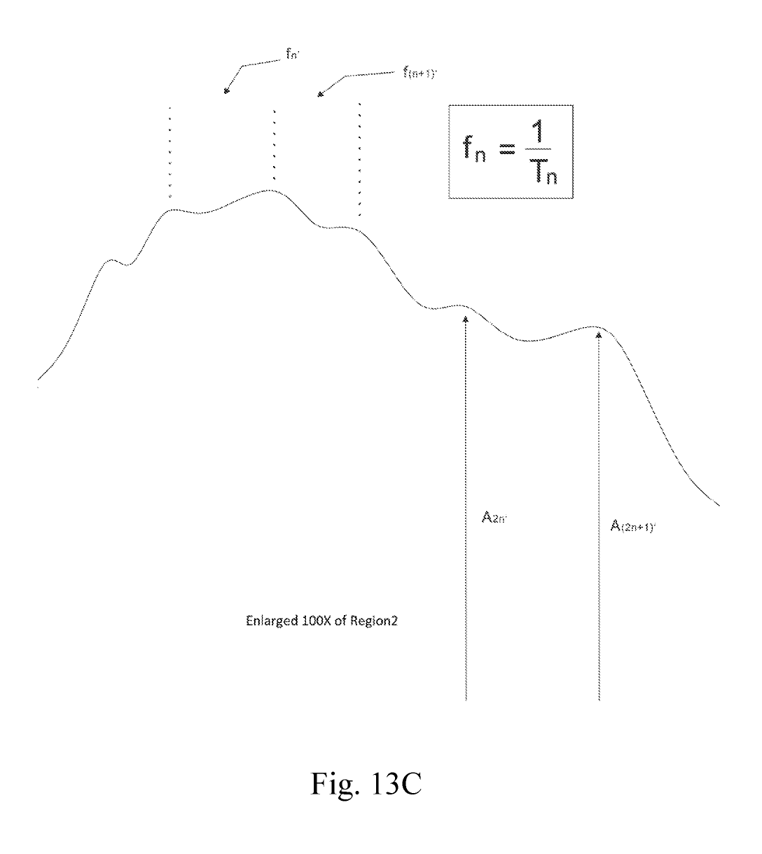

The electrical probes system of portable TES device is a pair of claw shape attachment (built-in with flexible PCB) (FIGS. 7 & 11) equipped with four pairs of electrodes. The probes system is turnable and removable (FIG. 9).

(a) There are two pairs of claw shape type probes performing the electrical stimulation on the Frontal Lobe area (see FIG. 7). They are the flexible and rotary attachments. Each pair can provide an angle of 250 (degree rotation) so it could match different sizes and shapes of human heads.

(b) One pair of electrodes is equipped especially for the Temporal Lobe area. It is the default electrodes and not movable.

(c) The other pair of electrodes is equipped for the stimulation on Cerebellum. It is a flexible attachment and can provide an angle of 120 (degree rotation) (FIG. 7).

Therefore, by using the special design electrode probes, users are freely to select the appropriate brain area performing the electrical stimulation.

The whole electronic circuitry is embodied into a detachable compartment as depicted in FIG. 10. Inside which it houses all the main functional units including Microprocessor, Memories, Power Management, Impedance Tracking System, Digital to Analog, Outputs Control, Micro-USB or USB-C Interface, Remote Control and a Rechargeable Battery Cell. The compartment is connected to the Probe system via two pairs of connections located in the inner sides of compartment and the main functional unit relative position. Output TES signals are sent out from the compartment's contacts to the electrodes ends inside the Probe system (FIG. 10).

Aside from the main unit of the device, it accompanies with a remote-control unit for handling the operation of the device when users are wearing it on their heads. The remote-control unit communicates with the main unit via 2.4 GHz wireless, Bluetooth, or infra-red wireless protocol. User's menu for selection of profile is shown inside the LCD display. The remote-control is also powered up by another set of rechargeable battery.

A simplified version of the portable TES system without remote-control is available. It is designed especially for the elderly patients and some patients with cognition disorders. The remote-control is no longer required, and NO LCD displayed will be available. The system is mainly operated by the built-in On/Off switch with colorful LED (different color lighting showing operation status) and buzzer (different beep sound indication) during the whole operating process. All other functions remain the same but only with a simple and easy operation by one-touch (On/Off Switch).

In the description of the specification of the present invention, the term “one embodiment” refers that described specific features, structure, materials or characteristics combined with this embodiment or example are included in at least one embodiment or example of the present invention. In this specification the illustrative description to the above term is not limited to the same embodiment or example. Moreover, described specific features, structure, materials or characteristics can be combined with each other in any one or more embodiments or examples.

The foregoing description is merely illustrative of the preferred embodiments of the present invention and is not intended to be limiting of the present invention, and various changes and modifications may be made by those skilled in the art. Any modifications, equivalent substitutions, improvements, and the like within the spirit and principles of the present invention are intended to be included within the protective scope of the present invention.

What is claimed is:

1. A non-invasive method, comprising using Composite Waveform as a basis of generating Transcranial Electrical Stimulation (TES) signals that provides significance improvements in penetration power into a brain at a “safely controlled voltage-current level”.

2. The method, as recited in claim 1, wherein: by using composite waveform, a stimulation profile is pre-programmed at combined frequencies, amplitudes and phase angles variations, which means the profile is customized according to needs, relevant profiles for individuals' needs are retrieved with and same TES signals are re-played again.

3. The method, as recited in claim 2, wherein: “ONE SESSION” of stimulation possessing various AC signals in different frequencies & amplitudes, DC signals as well by just one-time treatment profile, there is no interruption of applied signals due to change of profile like those traditional equipment.

4. The method, as recited in claim 3, wherein: improved penetration power is feasible due to high frequencies which is up to 40 kHz is enveloped inside low frequencies which is down to 0.001 Hz, together with amplitudes and phases variations, different profiles are theoretically simulated by means of numerical analysis and curving fitting techniques.

5. The method, as recited in claim 4, wherein: customized profiles for dedicated patients is stored in external devices, data containing the profiles are retrieved and downloaded back to the portable TES system device, customized profiles mean also for symptoms orientated, the same device is used to apply into different symptoms.

6. The method, as recited in claim 5, wherein: the TES system is designed as a battery-operated device and an operating power is small enough to operate for 2 hours (or longer) continuously once it is fully charged.

7. The method, as recited in claim 6, wherein: by using high resolutions of digital-to-analog amplifier to generate the stimulation signals together with smoothing techniques of curve fitting, spikes are removed away such that pain feelings of puncture or pricking is avoided.

8. The method, as recited in claim 7, wherein: an impedance measurement technique is employed into the hardware such that the stimulated current AND voltage levels are continuously monitored under safety magnitudes at below 35V and 10 mA, and therefore, the voltage-current characteristics are maintained within safety level without being affected by the factors due to changes in the state of brain. Impedance measurement can also detect whether the handset is wearing on or loosening off when one is applying onto the head.

9. The method, as recited in claim 8, wherein: the stimulation signals are spreading out through a delicate designed probes system, two sets of claw-shaped contact probes which contains electrodes that transmit stimulation signals to the dedicated brain's functional regions, one more set is dedicated for Temporal lobe, the claw shape of the contact probes increases effectiveness in transmitting the stimulation signals into and across the brain's functional regions and building up a globe type stimulation in good spatial distribution.

10. The method, as recited in claim 9, wherein: the direct attachment of probes system eliminated the needs of complicated and clumsy wirings from the equipment to the head like those traditional method.

11. The method, as recited in claim 10, wherein: the TES system is remotely accessed and controlled by means of a remote control.

12. The method, as recited in claim 11, wherein: the claw-shaped attachments in the probes system are rotational in two sets of angles, one set is for frontal lobe which allows +/25 degrees of adjustment, the other is +/−12 degrees for adjustment; both provided adjustments is allowed for individuals to adjust a good wearing so that the electrodes contacts can be firmly held onto the heads through these the claw-shapes design.

13. The method, as recited in claim 12, wherein: the claw-shaped attachments are designed such that they can be turnable and removable for cleansing purpose and replacement of sponges.

14. The method, as recited in claim 13, wherein: there are 8 pairs of electrodes residing in the probes system of the headset, when the stimulation signals are sending to the electrodes in a commutative manner, a complex network of conducting pathways will be developed in 3D manner over the entire stimulated brain regions.

15. The method, as recited in claim 14, wherein: the electrical conductivity of electrodes is further enhanced by introducing the use of brine water with the substrate of sponge and non-metallic conductive silicon rubber.

16. The method, as recited in claim 1, wherein: a main functional unit is detachable for the sake of convenience of maintenance, data retrieval and download.

17. The method, as recited in claim 16, wherein: in order to exert a significance bending inward force for holding the headset in position and hence ensure a good connectivity of electrodes onto the head, a strong bended stainless steel has put inside across the headband of the TES system's headset.

18. The method, as recited in claim 17, wherein: due to the method of using advanced electronic components like high resolutions of Analog to Digital device, ARM core microprocessor, flash memories, wireless and low current consumption of components, the whole TES system is designed in a good balance and light in weight and put in the center of gravity of a symmetric human being.

19. The method, as recited in claim 18, wherein: the portable TES system can be operated by a remote-control with displayed menu and instructions for ordinary patients, or be operated by ONE-TOUCH On/Off Switch showing operation process by LED lighting and beep sound without any remote-control for the patients with cognition disorders.
